(19) United States
(12) Patent Application Publication
(10) Pub. No.: US 2021/0251600 A1
(43) Pub. Date: Aug. 19, 2021

(54) ULTRASONIC DIAGNOSTIC APPARATUS, METHOD, AND PROGRAM

(71) Applicant: Hitachi, Ltd., Tokyo (JP)

(72) Inventors: Kazumasa ISE, Tokyo (JP); Seiji OYAMA, Tokyo (JP)

(21) Appl. No.: 17/082,458

(22) Filed: Oct. 28, 2020

(30) Foreign Application Priority Data
Feb. 18, 2020 (JP) 2020-024973

(51) Int. Cl. A61B 8/06; A61B 8/02; A61B 8/08

(52) U.S. Cl. CPC A61B 8/065; A61B 8/5207; A61B 8/488; A61B 8/02

(57) ABSTRACT

A computing device generates a Doppler waveform based on a signal obtained by transmitting and receiving an ultrasonic wave; applies a second-order differentiation process to the generated Doppler waveform; and identifies peak detection periods that have a polarity corresponding to a curve of a detection target peak. The polarity is of a second-order differential value obtained by the second-order differentiation process. The computing device further obtains, for each of the multiple identified peak detection periods, a peak scale based on a duration of the peak detection period and a peak value of the Doppler waveform in the peak detection period, and detects a peak in the Doppler waveform based on the peak scale corresponding to each of the multiple peak detection periods.

### CROSS REFERENCE TO RELATED APPLICATION

This application claims priority to Japanese Patent Application No. 2020-024973 filed on Feb. 18, 2020, which is incorporated herein by reference in its entirety including the specification, claims, drawings, and abstract.

### TECHNICAL FIELD

The present disclosure relates to an ultrasonic diagnostic apparatus, method, and program, in particular, those relating to techniques to detect peaks in Doppler waveforms.

### BACKGROUND

Ultrasonic diagnostic apparatuses are widely used. Some ultrasonic diagnostic apparatuses operate in a Doppler mode to measure a velocity of blood flow of a subject. In the Doppler mode, the velocity of blood flow along outgoing and incoming directions of ultrasonic waves is indicated with a Doppler waveform by measuring a Doppler shift frequency of ultrasonic waves that are transmitted to and reflected from the subject.

In general, the Doppler waveform is defined as a time waveform using a horizontal axis as a time axis and a vertical axis as a Doppler shift frequency axis (velocity axis). When the Doppler waveform is displayed as an image in which multiple pixels are aligned vertically and horizontally, each pixel value of vertically-aligned multiple pixels (pixel line) for a certain time point shows a frequency spectrum on the Doppler shift frequency axis. In other words, the Doppler waveform is represented with multiple pixel lines, each of which extends along the Doppler shift frequency axis, and the multiple pixel lines are arranged one after another along the time axis.

JP H7-241291 A and JP H7-241290 A disclose generation of Doppler waveforms using an ultrasonic diagnostic apparatus. JP H7-241291 discloses techniques to obtain a threshold of a noise level based on a pixel value of a certain region in a Doppler waveform image, and trace the Doppler waveform of the image based on the obtained threshold. JP H7-241290 A discloses techniques to detect peaks of Doppler waveforms.

### SUMMARY

Regarding Doppler waveforms, diagnosis of a subject may be performed by using a peak value that indicates the height of a peak. For example, regarding the velocity of blood flow at the heart, diagnosis may be performed using peak values of the E wave and the A wave. However, peaks caused by noises may appear in Doppler waveforms, resulting in detection of non-target peaks. Although JP H7-241290 A discloses techniques to resolve such an issue, because the techniques are to avoid erroneous detection of noise peaks that change in position along a time axis with the elapse of time, noise peaks that have a certain temporal relationships with detection-target peaks may still be erroneously detected.

An object of the present disclosure is to avoid detecting non-target peaks in Doppler waveforms.

One or more aspects of the present disclosure provide a computing device that is configured to generate a Doppler waveform based on a signal obtained by transmitting and receiving an ultrasonic wave, apply a second-order differentiation process to the generated Doppler waveform, and identify multiple peak detection periods that have a polarity corresponding to a curve of a detection target peak. The polarity is of the second-order differential value obtained by the second-order differentiation process. The computing device is further configured to obtain, for each of the plurality of identified peak detection periods, a peak scale based on a duration of the peak detection period and a peak value of the Doppler waveform in the detection period, and detect a peak in the Doppler waveform based on the peak scale corresponding to each of the multiple identified peak detection periods.

According to one or more aspects of the present disclosure, detection of non-target peaks in Doppler waveforms can be avoided.

### BRIEF DESCRIPTION OF THE DRAWINGS

Embodiments of the present disclosure will be described based on the following figures, wherein:

FIG. 1 shows a configuration of ultrasonic diagnostic apparatus according to an embodiment of the present disclosure;

FIG. 2 represents a Doppler waveform that indicates a velocity of blood flow at a mitral valve of a heart;

FIG. 3 shows a Doppler waveform indicating a velocity of blood flow at a mitral valve of a heart that is actually measured;

FIG. 4 is a flowchart showing a flow of a peak detection process;

FIG. 5 is a diagram describing, with time waveforms, a peak detection process of a Doppler waveform;

FIG. 6 shows an actually measured Doppler waveform; and

FIG. 7 shows an actually measured Doppler waveform that indicates a velocity of blood flow at a mitral valve of a heart.

### DETAILED DESCRIPTION

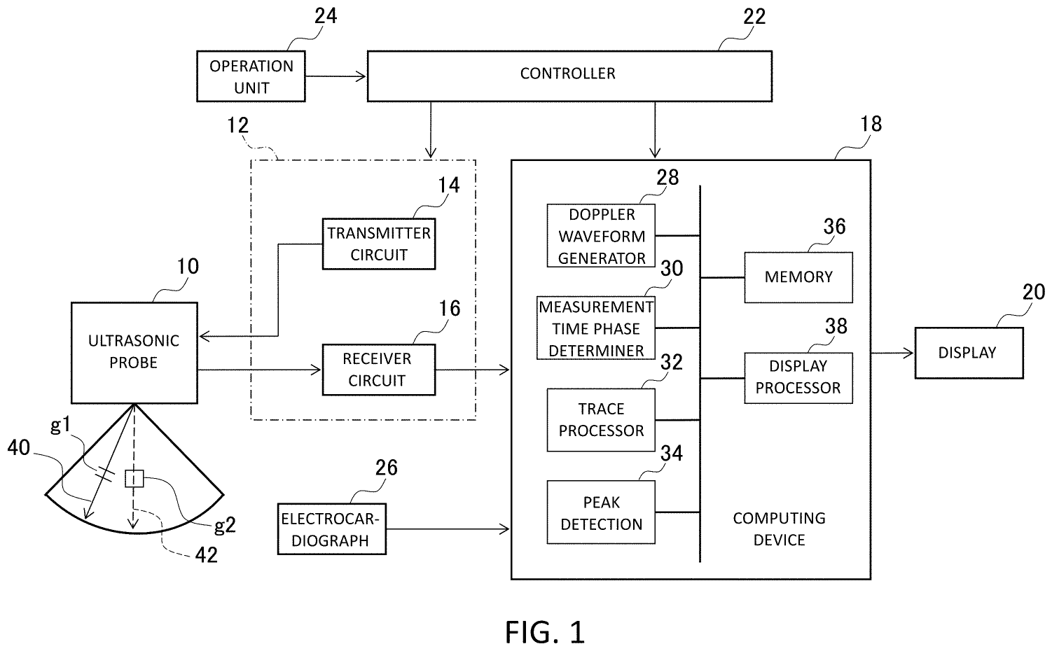

FIG. 1 shows a configuration of ultrasonic diagnostic apparatus according to an embodiment of the present disclosure. The ultrasonic diagnostic apparatus comprises an ultrasonic probe 10, a receiver transmitter 12, a computing device 18, a display 20, a controller 22, an operation unit 24, and an electrocardiograph 26. The operation unit 24 may include a keyboard, a mouse, a rotary knob, a lever, or other devices, to output, to the controller 22, operation instructions based on operations performed by a user. The controller 22 controls the ultrasonic diagnostic apparatus as a whole based on the operation instructions. The ultrasonic diagnostic apparatus is configured to operate in a Doppler mode, in which the receiver transmitter 12, the ultrasonic probe 10, the computing device 18, and the display 20 are controlled by the controller 22 as described below.

The receiver transmitter 12 includes a transmitter circuit 14 and a receiver circuit 16. The ultrasonic probe 10 includes multiple oscillating elements. The transmitter circuit 14 outputs outgoing pulse signals to each of the oscillating elements at a predetermined cycle. Each oscillating element converts the received outgoing pulse signals to ultrasonic pulses and transmits the ultrasonic pulses to a subject. The transmitter circuit 14 generates an outgoing beam in accordance with the ultrasonic pulses in a certain direction by adjusting delay time of pulse signals outputted to each oscillating element in order to strengthen the ultrasonic pulses emitted from each oscillating element in the certain direction.

Each oscillating element receives ultrasonic pulses reflected from the subject, converts the received ultrasonic pulses to incoming pulse signals, and outputs the obtained incoming pulse signals to the receiver circuit 16. The receiver circuit 16 generates incoming signals by phasing and adding the incoming pulse signals outputted from each oscillating element such that the incoming pulse signals based on the ultrasonic pulses received from the direction of the outgoing beam strengthen each other, and outputs the incoming signals to the computing device 18. Note that the receiver circuit 16 generates incoming signals for the ultrasonic pulses reflected from a diagnosis target region (hereinafter referred to as a “gate”) of the subject. Specifically, the receiver circuit 16 generates the incoming signals during a period from transmission of ultrasonic pulses to receipt of the ultrasonic pulses by each oscillating element after being reflected from the gate. Using this process, the receiver circuit 16 outputs, to the computing device 18, the incoming signals corresponding to the respective ultrasonic pulses that are transmitted in repeated cycles.

The computing device 18 may include a Doppler waveform generator 28, a measurement time phase determiner 30, a trace processor 32, a peak detection processor 34, a display processor 38, and a memory 36. The computing device 18 may be configured with a processor that executes programs stored in the memory 36 or an external storage medium to implement these elements (namely, the Doppler waveform generator 28, the measurement time phase determiner 30, the trace processor 32, the peak detection processor 34, and the display processor 38). The memory may store information that is to be used by the respective elements for computing, information to be temporarily stored in a computing process, information obtained as a result of computing, and other information.

The Doppler waveform generator 28 generates Doppler waveform data based on received signals which have been sequentially outputted from the receiver circuit 16 at repeated cycles. The Doppler waveform represented by the Doppler waveform data is defined as a time waveform using a horizontal axis as a time axis and a vertical axis as a velocity axis (Doppler shift frequency axis). When the Doppler waveform is displayed with vertically and horizontally aligned pixels, the pixel value of vertically aligned pixels (pixel line) for each time point represents a frequency spectrum shown along the Doppler shift frequency axis.

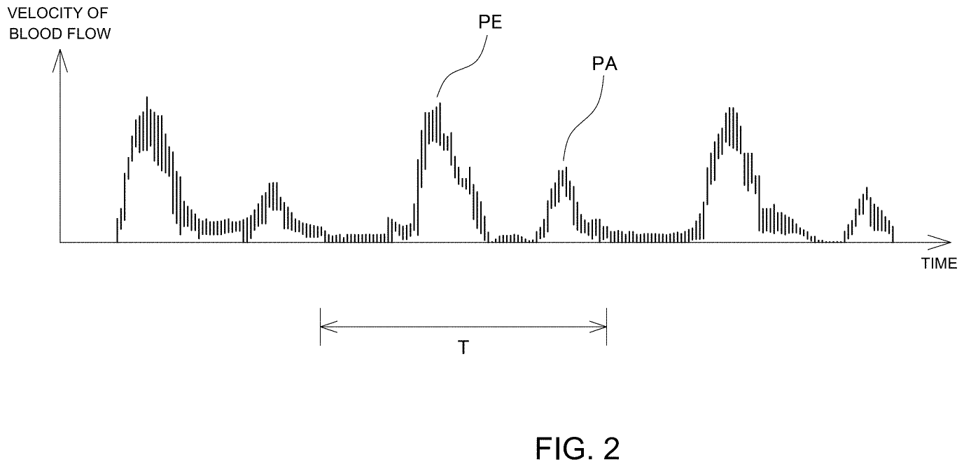

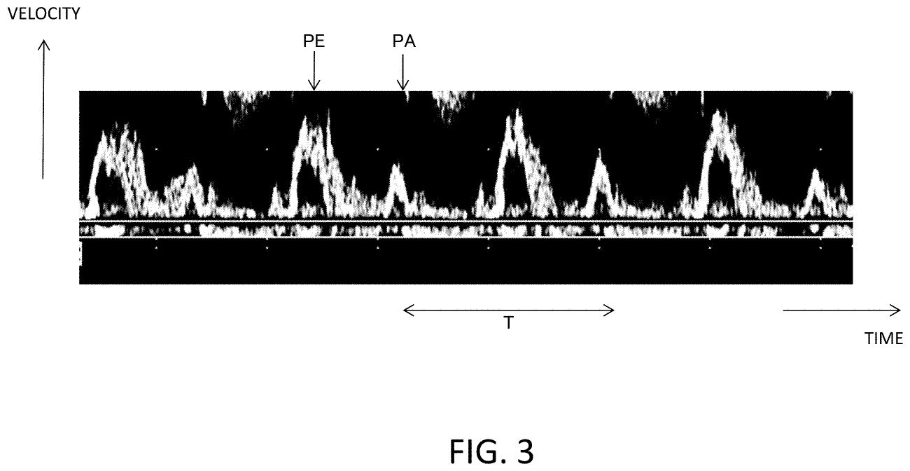

FIG. 2 shows a conceptual image of a Doppler waveform that represents the velocity of blood flow at a mitral valve of a heart. The horizontal axis represents time, whereas the vertical axis represents the velocity of blood flow. FIG. 3 shows a Doppler waveform that is actually measured regarding the velocity of blood flow at the mitral valve of the heart. FIGS. 2 and 3 show examples in which two peaks PE and PA appear in each heartbeat cycle T. The peak waveform that first appears in a single heartbeat cycle is called “E wave” and the subsequent peak is called “A wave”. In a diagnosis of a heart, the peak values of the E wave and the A wave are measured. The peak value is defined as the absolute value of the maximum value of the peaks in the time waveform.

As shown in FIG. 1, the ultrasonic diagnostic apparatus may generate two outgoing beams 40 and 42 in different directions using time divided processing. The ultrasonic diagnostic apparatus may obtain a Doppler waveform using time divided processing for a gate g1 set in the path of the outgoing beam 40 and a gate g2 set in the path of the other outgoing beam 42. Specifically, the ultrasonic diagnostic apparatus may obtain the Doppler waveform at the gate g1 set in the path of the outgoing beam 40 during a certain time period, and the Doppler waveform at the gate g2 set in the path of the other outgoing beam 41 during another time period.

The Doppler waveform generator 28 generates Doppler waveform data over multiple heartbeat cycles. The electrocardiograph 26 outputs, to the computing device 18, the heartbeat waveform data that represents the heartbeat waveform of the subject. The display processor 38 generates diagnostic image data representing Doppler waveforms and the heartbeat waveforms for the multiple heartbeat cycles and outputs the generated diagnostic image data to the display 20. The display 20 may be a liquid crystal display, an organic EL (electroluminescent) display, or any other display. The display 20 may be configured as a touch panel to be used with the operation unit 24. The display 20 displays diagnostic images representing the Doppler waveforms and the heartbeat waveforms for the multiple heartbeat cycles based on the diagnostic image data.

A peak detection process to detect peaks of Doppler waveforms is described below. The peak detection process may be performed when the ultrasonic diagnostic apparatus is in a freeze state, in which the image displayed on the display 20 freezes. In the freeze state, the Doppler waveform data and the heartbeat waveform data for the multiple heartbeat cycles are stored in the memory 36. In the freeze state, the receiver transmitter 12 may stop operations and the ultrasonic probe 10 may transmit no ultrasonic pulses.

The measurement time phase determiner 30 identifies a measurement time phase in accordance with user operations input through the operation unit 24. The measurement time phase is a heartbeat cycle in which a peak of a Doppler waveform is detected. Specifically, the measurement time phase determiner 30 checks the heartbeat waveform data stored in the memory 36 and identifies the measurement time phase from the multiple heartbeat cycles based on the control of the controller 22 in accordance with the operations input through the operation unit 24.

The trace processor 32 performs a trace processing for the Doppler waveform data corresponding to the determined measurement time phase. The trace processing is to obtain a Doppler waveform with reduced noise. The traced Doppler waveform data that represent the Doppler waveform with reduced noise are obtained by the trace processing. When the Doppler waveform is displayed by an image in which multiple pixels are aligned vertically and horizontally, the traced Doppler waveform data can be obtained by extracting pixels whose pixel values exceed a predetermined threshold.

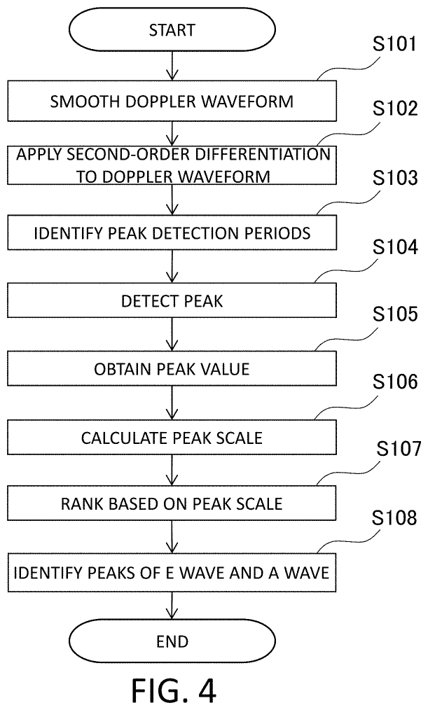

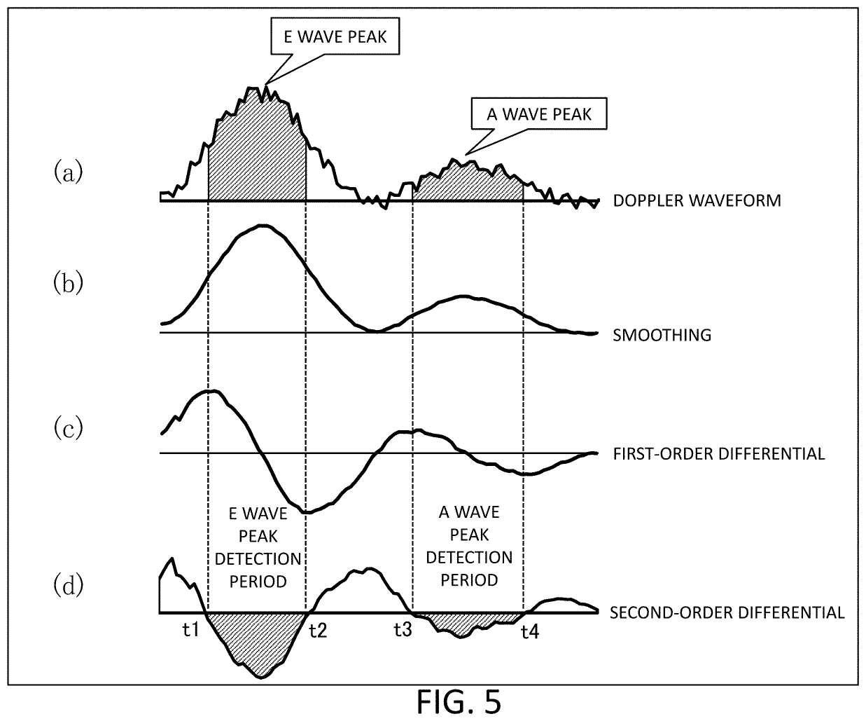

The peak detection processor 34 performs a peak detection process for the traced Doppler waveform data. The peak detection process obtains a position of a peak of the Doppler waveform along the time axis that is represented by the traced Doppler waveform data, and further obtains a peak value of the peak. FIG. 4 shows a flowchart that shows a flow of the peak detection process. FIG. 5 is a diagram that is used to describe the peak detection process for a Doppler waveform using an example of a time waveform. In FIG. 5, the horizontal axis and the vertical axis represent time and velocity respectively.

The peak detection processor 34 performs a smoothing process for the traced Doppler waveform data (S101). The smoothing process may be a moving-average process along the time axis. The moving-average process is a process to replace a value of the Doppler waveform in a time band of a certain time point with an average value of the Doppler waveform in a time band of a certain period of time including the time point. The time waveform (a) shown in FIG. 5 is a Doppler waveform before the smoothing process is applied, whereas the time waveform (b) shown in FIG. 5 is a Doppler waveform after the smoothing process is applied.

The peak detection processor 34 performs a first-order differentiation process to the smoothed Doppler waveform data, and further performs a second-order differentiation process (S102). The time waveform (c) shown in FIG. 5 is a first-order differential Doppler waveform that is obtained by the first-order differentiation process, whereas the time waveform (d) shown in FIG. 5 is a second-order differential Doppler waveform that is obtained by the second-order differentiation process. The first-order differential Doppler waveform shows a slope of the smoothed Doppler waveform.

The polarity of the second-order differential Doppler waveform represents whether the smoothed Doppler waveform forms a concave (an upward concave in the positive direction of the velocity axis) or a convex (a downward concave in the negative direction of the velocity axis). In a time period when the value of the second-order differential Doppler waveform is negative, the smoothed Doppler waveform forms a concave, whereas in a time period when the value of the second-order differential Doppler waveform is positive, the smoothed Doppler waveform forms a convex.

The peak detection processor 34 identifies a peak detection period in which the value of the second-order differential Doppler waveform is negative (S103). The peak detection processor 34 may identify multiple peak detection periods. In general, for a Doppler waveform representing the velocity of blood flow at a mitral valve of a heart, multiple peak detection periods are identified. In the time waveform (d) shown in FIG. 5, a time band between time t1 to time t2 and a time band between t3 to t4 are peak detection periods.

The peak detection processor 34 detects a peak of a detection target Doppler waveform in each peak detection period (S104). The peak detection processor 34 also obtains a peak value for the detected peak (S105), and calculates a peak scale (S106). The peak scale is larger for a longer peak detection period or a larger peak value of the smoothed Doppler waveform in the peak detection period. The peak scale may be defined, for example, as a product of the duration of the peak detection period and the peak value, as a time integrated value (area) of smoothed Doppler waveform in the peak detection period, or as an addition of the duration of the peak detection period and the peak value.

When multiple peak detection periods are identified, the peak detection processor 34 obtains the peak value and the peak scale of the smoothed Doppler waveform for each of the peak detection periods. The peak detection processor 34 ranks the peak detection periods in order of peak scale (S107) to identify the first peak detection period with the largest peak scale and the second peak detection period with the second largest peak scale.

The peak detection processor 34 identifies peaks of the E wave and the A wave (S108). Of the first peak detection period and the second peak detection period, the peak detection processor 34 obtains, as the peak position of the E wave, the peak position of the detection target Doppler waveform for the peak detection period of an earlier time phase, and also obtains, as the peak value of the E wave, the peak value that has been obtained in advance for the peak. Of the first peak detection period and the second peak detection period, the peak detection processor 34 obtains, as the peak position of the A wave, the peak position of the detection target Doppler waveform for the peak detection period of a later time phase, and also obtains, as the peak value of the A wave, the peak value that has been obtained in advance for the peak.

The peak detection processor 34 obtains an evaluation index based on the peak values of the E wave and the A wave. The evaluation index may be defined as, for example, a value obtained by dividing the peak value of the E wave by the peak value of the A wave. The peak detection processor 34 outputs, to the display processor 38, the evaluation index, the peak value of the E wave, and the peak value of the A wave. The display processor 38 causes the display 20 to display the evaluation index, the peak value of the E wave, and the peak value of the A wave.

In the above described peak detection process, the respective peaks of the E wave and the A wave are the detection target peaks, and the multiple peak periods having a negative second-order differential value of the smoothed Doppler waveform are identified. The peak scale is obtained for each peak detection period, and the respective peak detection periods corresponding to the E wave and the A wave are identified based on the ranked order of the peak scale. Further, the position and the peak value of the E wave are obtained in the peak detection period corresponding to the E wave. Similarly, the position and the peak value of the A wave are obtained in the peak detection period corresponding to the A wave.

A reason to identify the periods having a negative second-order differential value of a smoothed Doppler waveform as the peak detection periods is that the detection target peak forms a concave on the positive side (an upward concave) of the velocity axis. When the detection target peak forms a convex on the negative side (a downward concave) of the velocity axis, a time band having a positive second-order differential value of the smoothed Doppler waveform is identified as the peak detection period.

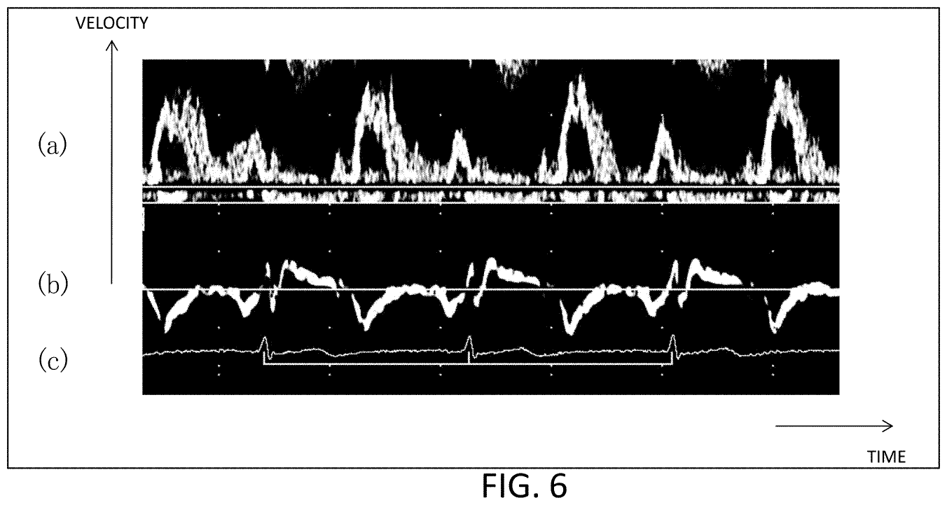

FIG. 6 shows actually measured Doppler waveforms. In FIG. 6, the waveform at the top is Doppler waveform (a) that represents the velocity of blood flow at a mitral valve of a heart; and the waveform in the middle is Doppler waveform (b) that represents the movement velocity of the mitral annulus of the heart. The top and middle Doppler waveforms (a) and (b) may be obtained using time divided processing based on two outgoing beams directed in different directions. The waveform at the bottom in FIG. 6 is heartbeat waveform (c) based on the heartbeat waveform data obtained by the electrocardiograph 26.

The respective peaks of the E wave and the A wave of the Doppler waveform that represents the movement velocity of the mitral annulus of the heart are convexes (downward concaves). In order to identify the peaks of the E wave and the A wave, the following processes are performed. In the peak detection process, multiple peak detection periods that have a positive second-order differential value of a smoothed Doppler waveform are identified. The peak scale is obtained for each of the identified peak detection periods, and the respective peak detection periods for the E wave and the A wave are identified based on the ranked order of the peak scale. Then, the position and the peak value of the E wave are obtained in the peak detection period corresponding to the E wave. Similarly, the position and the peak value of the A wave are obtained in the peak detection period corresponding to the A wave.

The peak detection processor 34 may also obtain a diastolic function that is defined as a ratio of the peak value of the E wave obtained from the velocity of the blood flow at a mitral valve to the peak value of the E wave obtained from the movement velocity of the mitral annulus. The peak detection processor 34 outputs, to the display processor 38, information indicating the positions of the peaks of the E wave and the A wave. The display processor 38 causes the display 20 to display information that indicates the positions of the respective peaks. The peak detection processor 34 may also output, to the display processor 38, not only the evaluation index, the peak value of the E wave, and the peak value of the A wave in accordance with the velocity of blood flow at a mitral valve, but also the evaluation index, the peak value of the E wave, the peak value of the A wave, and the diastolic function in accordance with the movement velocity of the mitral annulus. The display processor 38 causes the display 20 to display the respective values output from the peak detection processor 34.

Although the above embodiments are exemplary shown using two peaks as the detection target peaks, the detection target peak may be a single peak. In such a case, when multiple peak detection periods are identified, the peak position and the peak value may be obtained for the peak detection period that has the largest peak scale. Alternatively, the detection target peaks may be three or more peaks. In this case, the peak positions and the peak values are obtained for N number of peak detection periods in the ranked order from the largest peak scale where N is the number of detection target peaks.

The peak detection process is based on the following matters (1) to (5): (1) Doppler waveforms are generated based on signals obtained by transmitting and receiving ultrasonic waves. (2) The second-order differentiation process is applied to the Doppler waveforms. (3) Peak detection periods that have a second-order differential value obtained in the second-order differentiation process in a polarity corresponding to the curve of the detection target peak are identified. For example, when the detection target peak forms a concave, the time band having a negative second-order differential value is identified as the peak detection period, whereas when the detection target peak forms a convex, the time band having a positive second-order differential value is identified as the peak detection period. (4) A peak scale is obtained for each of the multiple peak detection periods. The peak scale is a value based on a duration of the peak detection period and the peak value of the Doppler waveform in the peak detection period. The peak scale may be larger for a longer duration of the peak detection period or a larger peak value of the Doppler waveform in the peak detection period. (5) The peak of the Doppler waveform is detected based on the peak scale corresponding to each of the peak detection period. Specifically, regarding a Doppler waveform for a single heartbeat, the peak of the Doppler waveform in the earlier peak detection period of the two peak detection periods that have top two peak scales is detected as the peak of the E wave, whereas the peak of the Doppler waveform in the remaining latter peak detection period is detected as the peak of the A wave.

The memory 36 that is a memory medium of the computing device 18 may store programs for executing the processes performed in methods in accordance with the above matters (1) to (5).

According to the peak detection process, the detection target peak is identified from multiple peaks in a Doppler waveform based on the peak scale in the respective peak detection periods that have been identified depending on whether the Doppler waveform forms a concave or convex. This can avoid erroneous detection of non-target peaks that have relatively small peak scales.

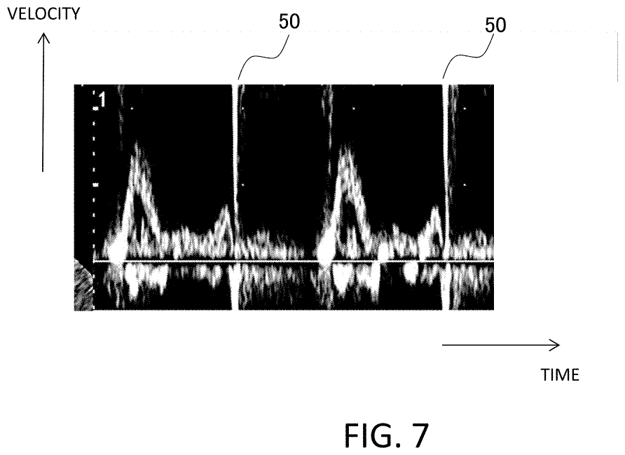

For example, as shown in FIG. 7, an impulse noise 50 may appear in a Doppler waveform that represents the velocity of blood flow at a mitral valve of a heart. The impulse noise 50 has a constant positional relationship with the E wave and the A wave along the time axis. The impulse noise 50 may occur when not only the blood flow but also tissue of the heart is located in the path of the outgoing beam. The peak detection processes according to the embodiments of the present disclosure can avoid erroneous detection of such an impulse noise as a detection target noise.

1. An ultrasonic diagnostic apparatus comprising:
a computing device configured to
generate a Doppler waveform based on a signal obtained by transmitting and receiving an ultrasonic wave;
apply a second-order differentiation process to the generated Doppler waveform;
identify a plurality of peak detection periods that have a polarity corresponding to a curve of a detection target peak, the polarity being of a second-order differential value obtained by the second-order differentiation process;
obtain, for each of the plurality of identified peak detection periods, a peak scale based on a duration of the peak detection period and a peak value of the Doppler waveform in the identified peak detection period; and
detect a peak in the Doppler waveform based on the peak scale corresponding to each of the plurality of peak detection periods.

2. The ultrasonic diagnostic apparatus according to claim 1, wherein
the peak scale is larger for a longer duration of the peak detection period or a larger peak value of the Doppler waveform.

3. The ultrasonic diagnostic apparatus according to claim 1, wherein
the peak scale is a value based on a time integrated value of the Doppler waveform in the peak detection period.

4. The ultrasonic diagnostic apparatus according to claim 2, wherein
the peak scale is a value based on a time integrated value of the Doppler waveform in the peak detection period.

5. The ultrasonic diagnostic apparatus according to claim 1, wherein
regarding a Doppler waveform for a single heartbeat, the computing device detects, of two of the peak detection periods that have top two peak scales, a peak of the Doppler waveform in a peak detection period in an earlier time phase as a peak of an E wave, whereas the computing device detects a peak of the Doppler waveform in the other peak detection period in a later time phase as a peak of an A wave.

6. The ultrasonic diagnostic apparatus according to claim 2, wherein
regarding a Doppler waveform for a single heartbeat, the computing device detects, of two of the peak detection periods that have top two peak scales, a peak of the Doppler waveform in a peak detection period in an earlier time phase as a peak of an E wave, whereas the computing device detects a peak of the Doppler waveform in the other peak detection period in a later time phase as a peak of an A wave.

7. The ultrasonic diagnostic apparatus according to claim 3, wherein
regarding a Doppler waveform for a single heartbeat, the computing device detects, of two of the peak detection periods that have top two peak scales, a peak of the Doppler waveform in a peak detection period in an earlier time phase as a peak of an E wave, whereas the computing device detects a peak of the Doppler waveform in the other peak detection period in a later time phase as a peak of an A wave.

8. The ultrasonic diagnostic apparatus according to claim 4, wherein
regarding a Doppler waveform for a single heartbeat, the computing device detects, of two of the peak detection periods that have top two peak scales, a peak of the Doppler waveform in a peak detection period in an earlier time phase as a peak of an E wave, whereas the computing device detects a peak of the Doppler waveform in the other peak detection period in a later time phase as a peak of an A wave.

9. An ultrasonic diagnostic method comprising:
generating a Doppler waveform based on a signal obtained by transmitting and receiving an ultrasonic wave;
applying a second-order differentiation process to the generated Doppler waveform;
identifying a plurality of peak detection periods that have a polarity corresponding to a curve of a detection target peak, the polarity being of a second-order differential value obtained by the second-order differentiation process,
obtaining, for each of the plurality of identified peak detection periods, a peak scale based on a duration of the peak detection period and a peak value of the Doppler waveform in the peak detection period, and
detecting a peak in the Doppler waveform based on the peak scale corresponding to each of the plurality of peak detection periods.

10. An ultrasonic diagnostic program to be executed by a computing device of an ultrasonic diagnostic apparatus, the ultrasonic diagnostic program causing the computing device to:
generate a Doppler waveform based on a signal obtained by transmitting and receiving an ultrasonic wave;
apply a second-order differentiation process to the generated Doppler waveform;
identify a plural of peak detection periods that have a polarity corresponding to a curve of a detection target peak, the polarity being of a second-order differential value obtained by the second-order differentiation process;
obtain, for each of the plurality of identified peak detection periods, a peak scale based on a duration of the peak detection period and a peak value of the Doppler waveform in the peak detection period; and
detect a peak in the Doppler waveform based on the peak scale corresponding to each of the plurality of peak detection periods.
